(19) United States
(12) Patent Application Publication
(10) Pub. No.: US 2015/0140527 A1
(43) Pub. Date: May 21, 2015

(54) Providing Interventions by Leveraging Popular Computer Resources

(71) Applicant: Microsoft Corporation, Redmond, WA (US)

(72) Inventors: Ran Gilad-Barach, Bellevue, WA (US); Pablo Enrique Paredes Castro, San Leandro, CA (US); Mary P. Czerwinski, Kirkland, WA (US); Paul R. Johns, Tacoma, WA (US); Ashish Kapoor, Kirkland, WA (US); Laura R. Pina, La Jolla, CA (US); Asta J. Roseway, Bellevue, WA (US); Kael R. Rowan, Kenmore, WA (US)

(21) Appl. No.: 14/084,524

(22) Filed: Nov. 19, 2013

(51) Int. Cl. A61B 5/16; A61B 5/11; G09B 5/00; A61B 5/00

(52) U.S. Cl. CPC A61B 5/165; A61B 5/6898; A61B 5/486; A61B 5/7264; A61B 5/0022; A61B 5/1112; G09B 5/00
USPC 434/236

(57) ABSTRACT

A computer system is described for providing intervention suggestion information to a user, for the purpose of changing a psychological state of the user. The information suggestion information identifies at least one recommended intervention, selected from a pool of candidate interventions. Each candidate intervention, in turn, involves a type of computer-related activity with which the user is likely already familiar. The computer system formulates the intervention suggestion information in the form of one or more messages, delivered to one or more user devices, such as a mobile user device, or a mobile user device in conjunction with an ambient presentation device. According to one optional aspect, the computer system chooses the recommended interventions based on context information. According to another aspect, the computer system selects interventions by adopting a particular balance between an exploitation mode and an exploration mode.

### BACKGROUND

Psychologists and other mental health professionals commonly formulate interventions for their patients. The interventions are intended to assist the patients in achieving desired psychological goals. For example, a professional may recommend one or more strategies that assist patients in combating a harmful psychological state, such as stress. More specifically, for instance, a psychologist may devise one or more techniques that help a parent in coping with the stress caused in interacting with a child having special needs. In other cases, the professional provides interventions to motivate a subject to perform a specific action, such as taking medication. The psychological state in this case corresponds to the patient's willingness or propensity to perform the desired action. However, for reasons that are not always well understood, interventions sometimes fail to achieve their intended goals. For instance, a patient often fails to adhere to a recommended course of therapy. Or if the intervention is performed, the patient may fail to reap its intended benefits.

### SUMMARY

A computer system is described herein for providing intervention suggestion information to a user, via one or more user devices, for the purpose of changing a psychological state of the user. The intervention suggestion information identifies at least one recommended intervention, selected from a pool of candidate interventions. Each candidate intervention in the pool, in turn, involves a general type of computer-related activity with which the user is likely already familiar. More formally stated, each candidate intervention in the pool of available interventions: (a) corresponds to a type of activity that has been performed using one or more computing devices for a purpose that may be independent of providing therapy; (b) corresponds to a type of activity that satisfies a prescribed popularity condition; and (c) maps to at least one therapy classification in a set of identified therapy classifications.

For example, the candidate interventions may be culled from activities performed using a social network system, a message-sending system (e.g., an Email system, an instant-messaging system, etc.), an online data storage system, a gaming system, a search system, and so on.

According to another illustrative aspect, the computer system may generate the intervention suggestion information based on context information. Among other items of information, the context information describes a contextual setting that applies to the user at the time that the intervention is provided. According to another implementation, the computer system may alternatively generate the intervention suggestion information without reference to user-specific context information, e.g., by generating the intervention suggestion information in a random manner, or based on context information that is not specific to the target user.

According to another illustrative aspect, the computer system delivers the intervention suggestion information to the user via a mobile user device, such as a smartphone. The intervention suggestion information may include a description of a recommended intervention, together with an activation mechanism for invoking the recommended intervention.

According to another implementation, the computer system may deliver the intervention suggestion information in the form of two messages. A first message provides an ambient presentation relating to a recommended intervention. A second message provides the ambient presentation in conjunction with explanatory content which describes the recommended intervention. The computer system may deliver the first message to a first user device, and deliver the second message to a second user device.

According to another illustrative aspect, the computer system is configured to choose the intervention suggestion information using a model, such as, but not limited to, a model produced using any machine learning technique. In one implementation, the model is configured to select a balance between an exploitation mode and an exploration mode. In the exploitation mode, the computer system is configured to select candidate interventions based primarily on their respective proven levels of relevance. In the exploration mode, the computer system is configured to select candidate interventions by favorably weighting candidate interventions as a positive function of their respective levels of uncertainty.

The above approach can be manifested in various types of systems, components, methods, computer readable storage media, data structures, graphical user interface presentations, articles of manufacture, and so on.

This Summary is provided to introduce a selection of concepts in a simplified form; these concepts are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a computer system for delivering intervention suggestion information to users.

FIG. 2 shows one particular non-limiting implementation of the computer system of FIG. 1.

FIG. 3 shows an illustrative mapping of computer-related activities to therapy classifications.

FIG. 4 shows one implementation of an intervention selection module, which is one component of the computer system of FIG. 1.

FIG. 5 shows an illustrative input vector produced by the intervention selection module of FIG. 4.

FIG. 6 shows an illustrative interaction flow that may be provided by the computer system of FIG. 1.

FIG. 7 shows one manner by which the computer system of FIG. 1 may deliver interaction suggestion information to two user devices, in the form of two respective messages.

FIG. 8 shows a procedure for selecting candidate interventions for inclusion in a pool of available interventions.

FIG. 9 shows a procedure which describes an overview of one manner of operation of the computer system of FIG. 1.

FIGS. 10 and 11 show two respective procedures by which the intervention selection module (of FIG. 4) may balance an exploitation mode with an exploration mode, in the course of selecting recommended interventions.

FIG. 12 shows a procedure which describes an interaction flow that may be provided by the computer system of FIG. 1.

FIG. 13 shows a procedure which represents an alternative mode of delivering intervention suggestion information to two user devices, in the form of two respective messages.

FIG. 14 shows illustrative computing functionality that can be used to implement any aspect of the features shown in the foregoing drawings.

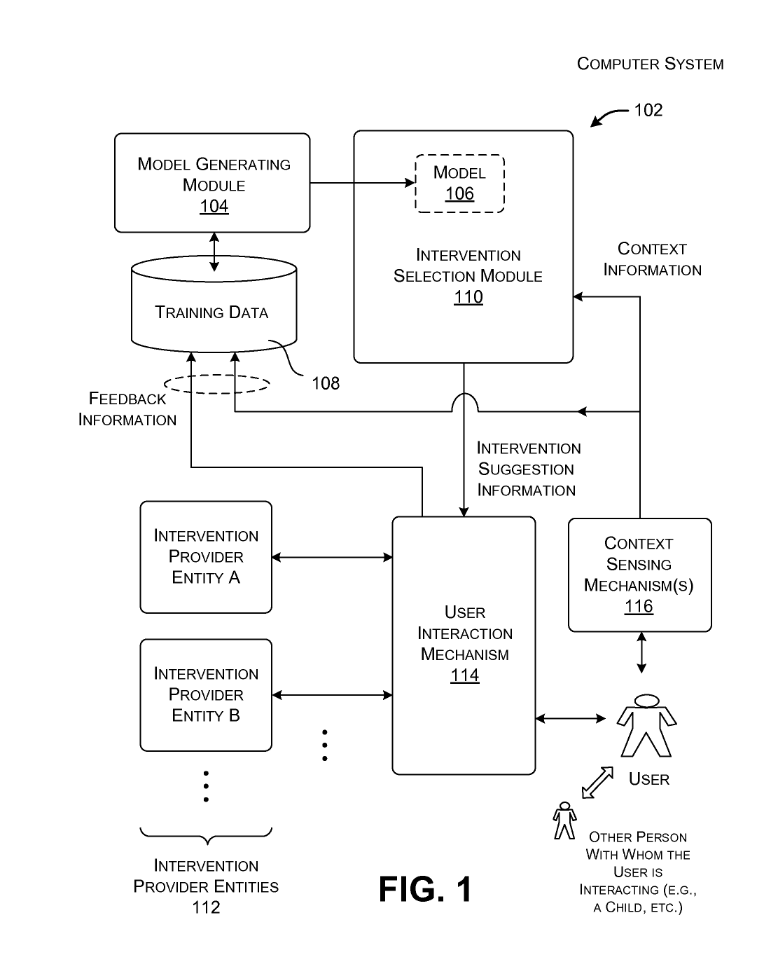

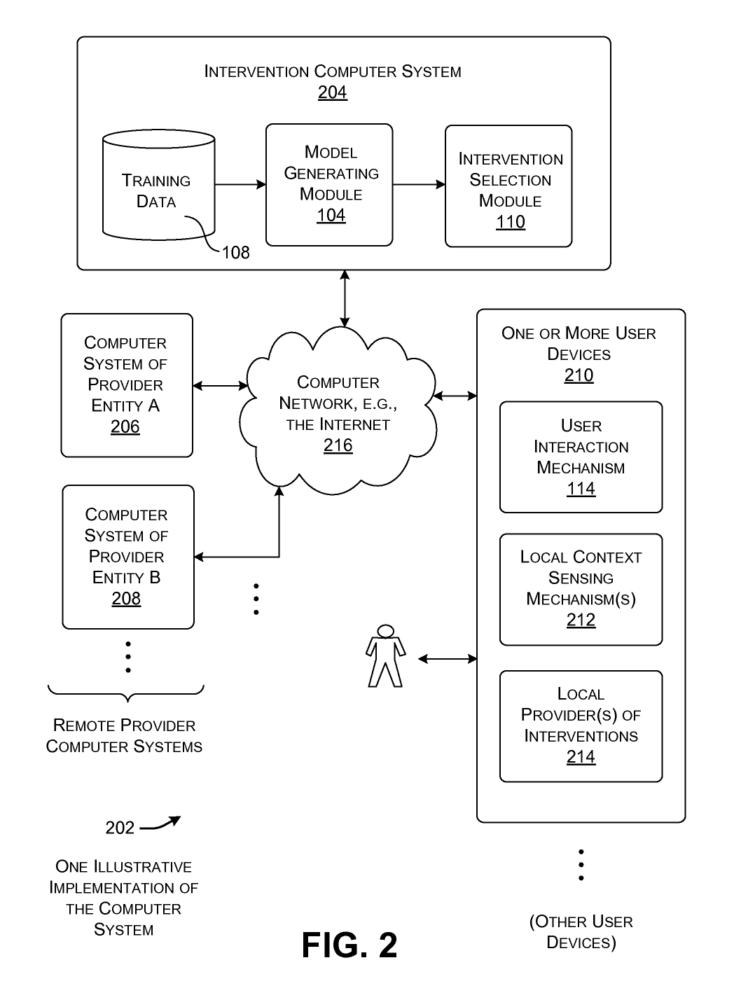

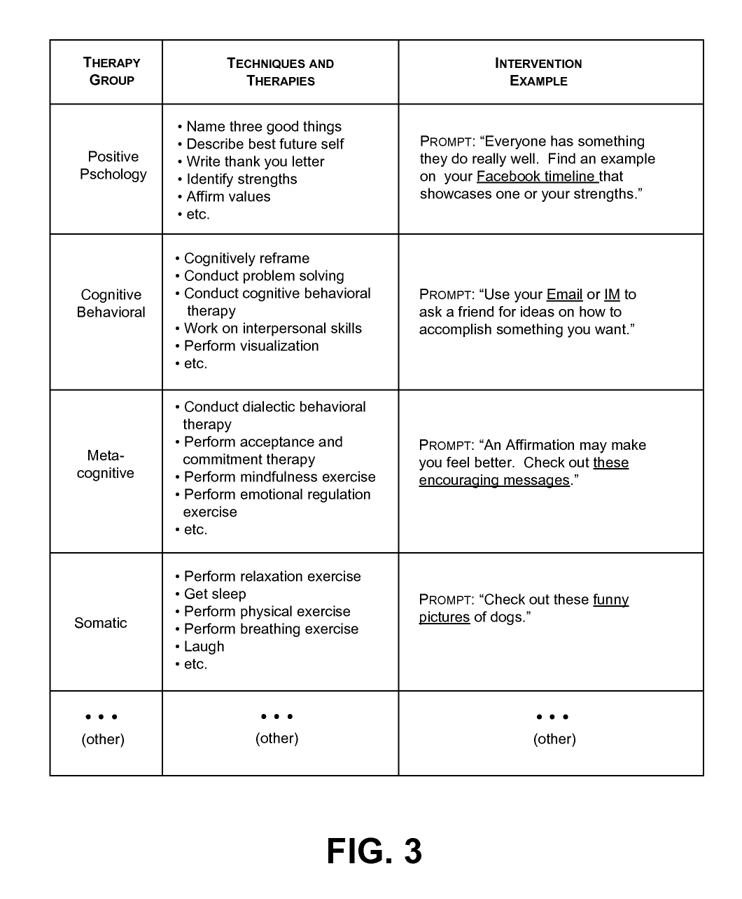

The same numbers are used throughout the disclosure and figures to reference like components and features. Series 100 numbers refer to features originally found in FIG. 1, series 200 numbers refer to features originally found in FIG. 2, series 300 numbers refer to features originally found in FIG. 3, and so on.

### DETAILED DESCRIPTION

This disclosure is organized as follows. Section A describes an illustrative computer system for selecting and delivering intervention suggestion information. Section B sets forth illustrative methods which explain the operation of the computer system of Section A. Section C describes illustrative computing functionality that can be used to implement any aspect of the features described in Sections A and B.

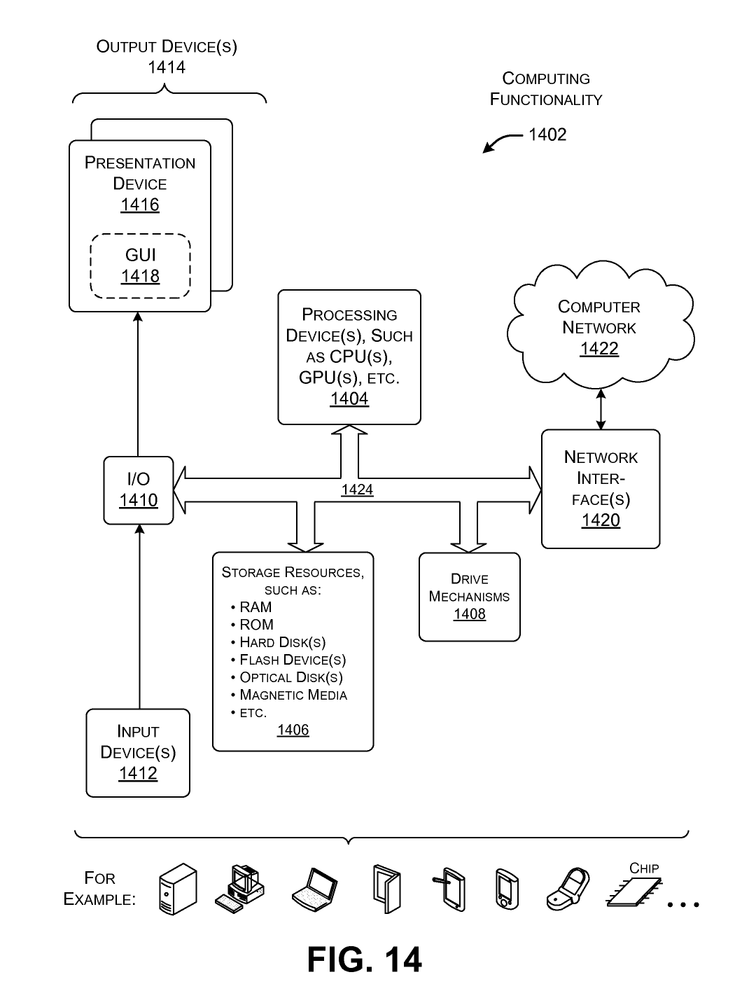

As a preliminary matter, some of the figures describe concepts in the context of one or more structural components, variously referred to as functionality, modules, features, elements, etc. The various components shown in the figures can be implemented in any manner by any physical and tangible mechanisms, for instance, by software running on computer equipment, hardware (e.g., chip-implemented logic functionality), etc., and/or any combination thereof. In one case, the illustrated separation of various components in the figures into distinct units may reflect the use of corresponding distinct physical and tangible components in an actual implementation. Alternatively, or in addition, any single component illustrated in the figures may be implemented by plural actual physical components. Alternatively, or in addition, the depiction of any two or more separate components in the figures may reflect different functions performed by a single actual physical component. FIG. 14, to be described in turn, provides additional details regarding one illustrative physical implementation of the functions shown in the figures.

Other figures describe the concepts in flowchart form. In this form, certain operations are described as constituting distinct blocks performed in a certain order. Such implementations are illustrative and non-limiting. Certain blocks described herein can be grouped together and performed in a single operation, certain blocks can be broken apart into plural component blocks, and certain blocks can be performed in an order that differs from that which is illustrated herein (including a parallel manner of performing the blocks). The blocks shown in the flowcharts can be implemented in any manner by any physical and tangible mechanisms, for instance, by software running on computer equipment, hardware (e.g., chip-implemented logic functionality), etc., and/or any combination thereof.

As to terminology, the phrase “configured to” encompasses any way that any kind of physical and tangible functionality can be constructed to perform an identified operation. The functionality can be configured to perform an operation using, for instance, software running on computer equipment, hardware (e.g., chip-implemented logic functionality), etc., and/or any combination thereof.

The term “logic” encompasses any physical and tangible functionality for performing a task. For instance, each operation illustrated in the flowcharts corresponds to a logic component for performing that operation. An operation can be performed using, for instance, software running on computer equipment, hardware (e.g., chip-implemented logic functionality), etc., and/or any combination thereof. When implemented by computing equipment, a logic component represents an electrical component that is a physical part of the computing system, however implemented.

The following explanation may identify one or more features as “optional.” This type of statement is not to be interpreted as an exhaustive indication of features that may be considered optional; that is, other features can be considered as optional, although not expressly identified in the text. Finally, the terms “exemplary” or “illustrative” refer to one implementation among potentially many implementations.

The functionality described herein can employ various mechanisms to ensure the privacy of user data collected and/or maintained by the functionality, in accordance with user expectations and applicable laws and norms of relevant jurisdictions. For example, the functionality can allow a user to expressly opt in to (and then expressly opt out of) the provisions of the functionality. The functionality can also provide suitable security mechanisms to ensure the privacy of the user data (such as data-sanitizing mechanisms, encryption mechanisms, password-protection mechanisms, etc.).

A. Illustrative Computer System

A.1. Overview of the Computer System

FIG. 1 shows a logical overview of a computer system 102 for delivering intervention suggestion information to users. As the term is used herein, an intervention corresponds to any tactic that the user may take to achieve any desired goal that has a bearing on the mental state of the user. For example, the intervention may aim to reduce a psychological state that is deemed harmful for any reason, such as stress, or more specifically, stress that may occur in the parenting of special needs children (e.g., children with attention deficit hyperactivity disorder). Alternatively, or in addition, the intervention may aim at promoting a psychological state that is deemed beneficial, such as the user's willingness or propensity to eat healthy food, or take medication. The above interventions are generally intended to improve the health of the users who perform the interventions. But in other cases, an entity may devise interventions for the primary purpose of achieving non-health objectives, such as by encouraging a user to take a particular action in the marketplace (such as by purchasing a particular item), adhere to an educational program, treat another person (such as a spouse) in a more respectful and loving manner, develop a jobs-related skill, and so on. In short, no limitation is placed on the nature of the interventions, the purpose of the interventions, the identity of the entity (or entities) which creates the interventions, or the identity of the user who is the target of the interventions.

In one implementation, the computer system 102 delivers the interventions to the user in a particular context. The context corresponds to a situation which is affecting the user at a current time, or otherwise relevant to the user at the current time. Context information describes the context. The context information includes information regarding the personality traits of the user, the current psychological state of the user, the setting in which the user is currently interacting with the computer system 102, and so on, or parts thereof. In some cases, the context information may indicate that the user is not currently interacting with another person. In other cases, the context information may indicate that the user is interacting with one or more other people, such as a child, a spouse, a co-worker, and so on.

This subsection presents an overview of the computer system 102. Later subsections provide additional information regarding selected aspects of the computer system.

To begin with, the computer system 102 includes a model generating module 104 that is configured to generate a model 106 based on training data maintained in a data store 108. The model generating module 104 may use any machine learning technique to generate the model 106, such as a technique selected from the domain of reinforcement learning. In a yet more particular implementation, the model 106 that is produced represents the task of selecting interventions as a contextual multi-arm bandit problem (to be described in greater detail in Subsection A.3). The training data in the data store 108 may describe salient aspects of previous interventions conducted by the computer system 102, including outcome information that indicates the degree of success of those interventions.

In one implementation, an intervention selection module 110 operates by receiving context information that describes the current context of the user. The intervention selection module 110 then uses the model 106 to map the context information to intervention suggestion information. The intervention suggestion information identifies one or more recommended interventions, selected from a pool of available candidate interventions. In another implementation, the intervention selection module 110 generates intervention suggestion information without reference to context information, or without reference to some items of context information (to be described below).

Generally, candidate interventions are selected for inclusion in the pool of prospective interventions if there is a reason to believe that they may benefit the user in achieving desired therapeutic goals. In some implementations, at least some interventions are expected to satisfy additional qualifying considerations.

For example, in one implementation, some or all of the candidate interventions correspond to types of computer-related activities with which the user is likely already familiar, outside the context of delivering therapy. More formally stated, each of these candidate interventions is selected for inclusion (or preferential weighting) in the pool of available interventions providing that it: (a) corresponds to a type of activity that has been performed using one or more computing devices, for a purpose that may be independent of providing therapy; (b) corresponds to a type of activity that satisfies a prescribed popularity condition which indicates that it is well known within a community of users; and (c) maps to at least one therapy classification in a set of identify therapy classifications.

In addition, or alternatively, each of at least some candidate interventions may be selected (or preferentially weighted) providing that it satisfies a prescribed simplicity consideration, e.g., by possessing a level complexity that is below a prescribed complexity threshold. Level of complexity can be measured in different ways, such as the amount of time it takes to complete the candidate intervention, and/or the number of operations associated with the candidate intervention, and/or the ability of a typical user to understand the candidate intervention, etc.

In those cases in which constraints are placed on some candidate interventions, at least some of the constraints may correspond to mandatory considerations. In one implementation, a candidate intervention which fails to satisfy a mandatory factor will not be placed in the pool of available candidate considerations. Alternatively, or in addition, at least some of the above factors correspond to preferred considerations. In one implementation, a candidate intervention which fails to satisfy a preferred factor will be discounted in an appropriate manner (such as by negatively weighting this intervention), yet will still be included in the pool of available candidate interventions. Subsection A.2 (below) provides additional information regarding illustrative considerations that go into selecting candidate interventions.

More concretely stated, many of the candidate interventions may involve types of computer-related activities that users engage in throughout the day for reasons unrelated to the delivery of therapy, via commonly-used computing devices, such as smartphones, tablet-type devices, etc. Different intervention providers 112 may provide the resources used in performing these candidate interventions. For example, some candidate interventions may involve actions that a user performs while interacting with a social network system, such as the social network systems provided by Facebook Inc. of Menlo Park, Calif., or Twitter, Inc. of San Francisco, Calif., etc. Other candidate interventions may involve actions that a user performs while interacting with a message-sending system, such as an Email system, an instant messaging system, etc. Other candidate interventions may involve actions that a user performs while interacting with a calendar system. Other candidate interventions involve actions that a user takes while interacting with a data storage system, such as a system which stores text documents, static images, videos, songs, etc. Other candidate interventions involve actions that the user may perform while playing a computer game. Other candidate interventions may involve actions that a user performs while interacting with a search system. The above examples are cited by way of illustration, not limitation.

The user may engage in some of the above-identified interventions in an online fashion, e.g., by using one or more user devices to interact with particular websites or web services, and/or one or more remote user devices operated by other users. The user may engage in other interventions in a mostly offline fashion, e.g., by using a local game console or handheld game device to play a game. In other cases, the user may perform some aspects of an intervention by interacting with remote computer functionality and other aspects of an intervention by interacting with local computer functionality. More generally, an intervention is said to involve or use computer-related resources insofar as the user uses one or more computers to learn about and/or conduct the intervention.

A user interaction mechanism 114 provides functionality by which the user may interact with the intervention selection module 110. Different events may initiate this interaction. In one case, the user may use the user interaction mechanism 114 to expressly request the intervention selection module 110 to deliver interaction suggestion information. Alternatively, or in addition, a context sensing mechanism 116 may continually (or periodically) supply context information to the intervention selection module 110 that reflects the current psychological state of the user. That context information may prompt the intervention selection module 110 to begin preparing intervention suggestion information. For example, the context information may indicate that the user is likely undergoing a high degree of stress at the present moment, prompting the intervention selection module 110 to begin preparing the intervention suggestion information.

Alternatively, or in addition, the intervention selection module 110 may deliver intervention suggestion information based on other considerations, such as by delivering interventions when the user performs certain actions (such as by unlocking a screen or opening an application). Alternatively, or in addition, the intervention selection module 110 may deliver interventions according to a fixed schedule or in a random manner. Still other factors may trigger the intervention selection module 110 to generate the intervention suggestion information, as set forth in Subsection A.4.

Once triggered, the intervention selection module 110 can optionally collect additional context information which describes the user's current context. As part of that collection task, the user interaction mechanism 114 may optionally ask the user to perform a self-assessment of his or her psychological state. In one implementation, the intervention selection module 110 then formulates an input vector (or other representation of context) having features values which represent the context information. Next, the intervention selection module 110 uses the model 106 to map to the input vector to one or more recommended interventions that are likely to be helpful to the user in achieving desired goals. The intervention selection module 110 then formulates intervention suggestion information that describes the recommended interventions and sends the intervention suggestion information to the user interaction mechanism 114.

More specifically, in one case, the intervention selection module 110 formulates the intervention suggestion information as a message that describes one or more recommended interventions, together with an optional activation mechanism (e.g., a hyperlink or the like) which allows the user to activate the recommended intervention. In one implementation, the intervention selection module 110 may deliver this message to a single user device, such as a mobile user device (e.g. a smartphone or the like).

In another case, the intervention selection module 110 formulates the intervention suggestion information as two messages. A first message provides an ambient presentation relating to a recommended intervention. A second message provides the ambient presentation in conjunction with explanatory content which describes the recommended intervention (and optionally provides an activation mechanism by which the user may invoke the intervention). The intervention selection module 110 may deliver the first message to a first user device and deliver the second message to a second user device.

Upon receipt of the intervention suggestion information, the user may optionally invoke an intervention by clicking on or otherwise selecting the activation mechanism associated with the intervention. A corresponding intervention provider entity then provides resources for use in performing the intervention. As a final operation in the interaction flow, the user interaction mechanism 114 may optionally ask the user to assess his or her psychological state, after having performed the recommended intervention. Subsection A.4 provides additional details regarding the above-summarized interaction flow.

The model generating module 104 is configured to receive feedback information over the course of the user's interaction with the computer system 102. The feedback information may include assessment information supplied by the user before and after the intervention is performed (if that assessment information is collected). The feedback information can also optionally include any other context information which describes the specific circumstance of the user and/or other contextual considerations, before, during, and/or after the delivery of the intervention suggestion information. The model generating module 104 may use the feedback information generate a new model. More specifically, the model generating module 104 may update the model based on any timing consideration, such as by updating the model on a periodic basis, and/or updating the model on an event-driven basis, e.g., by updating the model when a prescribed amount of feedback information has been received.

Overall, a number of factors may contribute to the success of the interventions recommended by the computer system 102. First, each of the candidate interventions in the pool of candidate interventions leverages commonly-used computer-related resources. The user's presumed familiarity with these resources increases the probability that the user will perform the recommended interventions. Second, in one implementation, the computer system 102 intelligently selects from among the candidate interventions based on context information. This aspect potentially increases the relevance of the recommended interventions with respect to a particular user, further increasing the chances that the user will perform the recommended interventions. Still other factors may contribute to the success of recommended interventions provided by the computer system 102.

FIG. 2 shows one particular non-limiting implementation 202 of the computer system 102 of FIG. 1. This implementation 202 is described by way of illustration, not limitation. As will be described, other implementations may be used to implement the logical functions described with reference to FIG. 1.

In the implementation of FIG. 2, an intervention computer system 204 may implement at least the model generating module 104, the data store 108 (which stores the training data), and the intervention selection module 110. The intervention computer system 204, for example, may be implemented by one or more server computing devices, in conjunction with one or more data stores and/or other computer equipment. For instance, the intervention computer system 204 may be implemented as a cluster of one or more server computing devices, which together implement a cloud computing platform. The functionality provided by the intervention computer system 204 may be provided at a single site or distributed over two or more sites.

One or more other computer systems may implement the services of the entities which provide the interventions. These provider computer systems, for instance, include a provider computer system 206 of provider entity A, a provider computer system 208 of provider entity B, and so forth. Each such provider computer system may be implemented in the manner stated above, e.g., by one or more server computing device, in conjunction with one or more data stores and/or other computer equipment. The functionality associated with each such provider computer system may be provided at a single site or distributed over plural sites.

A user may interact with the implementation 202 via one or more user devices 210. Any such user device may correspond to a mobile user device or a (traditionally) stationary user device. For example, the one or more user devices 210 may include one or more of: a smartphone or other cellular telephone device; a media-playing device; an electronic book-reader device; a portable digital assistant device; a stylus-type computing device; a portable game console device; a tablet-type computing device; a workstation computing device; a laptop computing device; a game console device; a set-top box device; a special-purpose computing device (particularly designed for the delivery of interventions); a wearable computing device, and so on. These examples are cited by way of illustration, not limitation; the user devices 210 may encompass yet other types of computing devices.

In one implementation, the user devices 210 may include the user interaction mechanism 114, described above. To repeat, the user interaction mechanism 114 provides an interface through which a user may interact with the intervention selection module 110, e.g., by optionally entering self-assessment information, receiving intervention suggestion information, etc.

The user devices 210 may also include any local context sensing mechanisms 212. The local context sensing mechanisms 212 describe the context in which each intervention is generated. Some of the local context sensing mechanisms 212 may be integrated into the housing of one or more of the user devices 210. Alternatively, or in addition, some of the local context sensing mechanisms 212 may be separate from, but communicatively coupled to, the user devices 210. Alternatively, or in addition, some of the local context sensing mechanisms 212 may be neither physically associated with, nor communicatively coupled to, the user devices 210, but nonetheless are provided in proximity to the user devices 210. Although not shown, other context sensing mechanisms may be provided at a remote location with respect to the user. Further, some of the local context sensing mechanisms 212 may provide their services in conjunction with functionality provided by remote systems.

The local context sensing mechanisms 212 may include different types of mechanisms, selected from the following representative and non-exhaustive list of sensing mechanisms:

Position-determining devices. The local context sensing mechanisms 212 may include position-determining devices, such as any of a Global Positioning System (GPS) mechanism, a triangulation mechanism, a dead-reckoning mechanism, and so on.

Motion-sensing devices. In addition, or alternatively, the local context sensing mechanisms 212 may include motion-sensing devices, such as any of one or more accelerometers, one or more gyroscopes, and so on.

Physiological sensing mechanisms. In addition, or alternatively, the local context sensing mechanisms 212 may include any type of sensor mechanism which captures the physiological state of the user, such as electrodermal sensing mechanisms, blood pressure sensing mechanisms, pulmonary sensing mechanisms, brain activity sensing mechanisms, and so on.

Voice detection mechanisms. In addition, or alternatively, the local context sensing mechanisms 212 can include mechanisms which capture and analyze the voice of the user. For example, some local context sensing mechanisms 212 can apply known filters to the user's voice signal to detect the presence of stress that may be affecting the user. Other semantic-based sensing mechanisms can apply voice recognition to the user's voice signal, converting the voice signal into textual content. Such local context sensing mechanisms 212 may then determine whether the user has uttered any keywords or phrases which correlate with certain psychological states.

Image and video detection mechanisms. In addition, or alternatively, the local context sensing mechanisms 212 can include image and video recognition mechanisms for capturing and analyzing the visual appearance of the user. For example, some local context sensing mechanisms 212 can apply known techniques to recognize the facial expression or gaze of the user, and to correlate that recognition result with particular psychological states. Other local context sensing mechanisms can determine the static posture of the user and/or gestures performed by the user, and correlate that recognition result with psychological states. For example, the local context sensing mechanisms 212 can use depth camera technology to generate a three-dimensional representation of the user's body (or a part of the user's body), and then compare that representation with telltale posture or gesture information that is associated with particular psychological states. The depth-camera technology may be implemented using a structured light technique, a stereoscopic technique, or some other technique. One commercial system for generating and analyzing depth images is the Kinect™ system, providing by Microsoft Corporation of Redmond, Wash.

Device interaction sensing mechanisms. Alternatively, or in addition, the local context sensing mechanisms 212 can include mechanisms for determining the manner in which the user is interacting with computer equipment, such as the manner in which the use is interacting with the user devices 210. For example, the local context sensing mechanisms 212 can determine the pressure with which the user is typing on a keyboard, or interacting with a touch-sensitive screen, or interacting with mouse device, etc.

The above examples of context sensing mechanisms are cited by way of illustration, not limitation. Other implementations can incorporate yet other types of sensing mechanisms not mentioned above, or omit one or more sensing mechanisms mentioned above. Further, as will be described below, other context sensing mechanisms can, while complying with applicable privacy considerations, extract additional user-related context information by examining information associated with the user, maintained by any application, website, service, account, etc. For example, these other context sensing mechanisms can extract information about the user from a calendar application. Other context sensing mechanisms can detect conditions associated with the general environment in which the user operates, such as conditions pertaining to the weather, time of the year, time of the day, financial markets, news-related events, and so on.

FIG. 2 indicates that the user devices 210 may also include one or more local providers of interventions 214, in addition to the remote provider computer systems (206, 208) described above. A local provider of an intervention may correspond to functionality that provides an intervention experience to the user, and which is local relative to the user devices 210. For example, a local provider of an intervention may correspond to an application that is installed on the user devices 210. In still other cases a remote provider (such as a web service) may work in conjunction with a local provider (such as a smartphone app) to provide a particular intervention.

A computer network 216 communicatively couples all or some of the above-identified components together. The computer network 216 may correspond to a local area network, a wide area network (such as the Internet), point-to-point communication links, etc., or any combination thereof.

As a closing comment, note that the delegation of functions to particular devices in FIG. 2 is described by way of illustration, not limitation. The delegation can be modified in any manner. For example, one or more functions performed by the intervention computer system 204 can be performed, in whole or in part, by the user devices 210, and vice versa.

A.2. Functionality for Selecting Candidate Interventions

As summarized above, the intervention selection module 110 chooses from among a pool of available candidates interventions, to provide one or more recommended interventions. The following explanation clarifies one technique for initially producing some or all of the candidate interventions in the pool candidate interventions. In one implementation, an administrator or other individual (or team of individuals) may manually apply the technique to choose the candidate interventions. In another implementation, an automated agent may automate the selection of candidate interventions, e.g., by automatically or semi-automatically identifying the characteristics of each candidate intervention, and then determining whether those characteristics satisfy stated criteria.

As a first criterion, the selection technique aims to find computer-related activities that have therapeutic effects associated with one or more therapy classifications, for a particular therapeutic goal under consideration. For example, FIG. 3 identifies four representative categories of therapies that may be helpful in reducing a user's level of stress (in a first column). FIG. 3 also lists examples of therapies associated with each class (in a second column). As a point of clarification, certain therapies may fall under two or more classifications, but are associated with a single classification in FIG. 3 to simplify explanation.

A “positive psychology” classification describes a set of techniques aimed at focusing the user's attention on factors which contribute to well-being. For example, one technique in this category asks the user to identify positive events in his or her life. Another technique asks the user to write a thank you letter to express gratitude to some real or fictional person or group, and so on. The last column of FIG. 3 identifies an activity that the user may perform using familiar social-networking tools that conforms to the positive psychology classification. That technique instructs the user to access his or her Facebook page and identify a logged event which showcases a personal strength of the user.

As a point of clarification, the specific act of accessing a social network page and attempting to locate logged events which reflect positively on the user may not be a common task for the user, as stated that level of specificity. But the general type of task of accessing a social network page and reviewing entries is likely to be a familiar task to many users, and is likely to have been performed for non-therapy-related reasons. It is in this general sense that this type of activity can be said to have traditionally served a familiar pre-existing purpose that is independent of providing therapy. And as mentioned above, an intervention can be said to use or involve computer-related resources insofar as those resources are involved in learning about and/or conducting the intervention.

A “cognitive behavioral” category describes techniques which encourage the user to explore the cognitive component of negative psychological states, e.g., by identifying the triggers of his or her thoughts, and then challenging the appropriateness of those thoughts. One such technique in this category asks the user to engage in problem solving, with the objective of finding a solution to a typical situation that leads to a stressful state. The last column of FIG. 3 identifies an activity that the user may perform using familiar message-sending tools that conforms to this classification. That technique instructs the user to send an Email message to a friend, asking the friend how the user might accomplish a desired goal. Again, this type of activity is considered a familiar task because the user may frequently send Email messages to his or her friends (although the specific activity of writing to a friend to ask for help in solving a problem may not necessarily correspond to familiar task.)

A “meta-cognitive” category encompasses techniques that aim to combat a psychological problem by providing an appropriate emotional response to the problem. One such technique in this category asks the user to perform an exercise directed at regulating his or her emotion. Another technique helps the user emotionally accept a certain situation, and so on. The last column of FIG. 3 identifies an activity that the user may perform using a familiar data storage website that conforms to this therapy classification. That activity asks the user to access and interact with a website that provides a list of affirmative messages, or a randomly-chosen affirmative message.

A “somatic” category encompasses physical activities that the user may perform to achieve a desired change in psychological state. Such techniques may, for instance, encourage the user to sleep, relax, exercise, laugh, breath in a certain manner, etc. The last column of FIG. 3 identifies an activity that the user may perform that conforms to the somatic classification, using a familiar information-retrieval website. That activity asks the user to visit the website to see one or more comical images, with the objective of inducing laughter.

The four categories (and associated computer-related techniques) described above are cited by way of illustration, not limitation. Other classifications and techniques may be appropriate for other implementations and/or for other therapeutic goals being sought.

FIG. 3 shows that each instance of intervention suggestion information may be formulated as at least one message. That message may include text content (and/or other media content) that describes the intervention. The intervention suggestion information may optionally also include an activation mechanism by which the user may invoke the intervention. In the case of FIG. 3, the activation mechanism corresponds to a hyperlink that is embedded within the text content. But in another case, the activation mechanism may correspond to a separate URL (or other link) to the entity which provides the intervention. In another case, the computer system 102 automatically invokes one or more recommended interventions upon their delivery, or some time thereafter.

As set forth in Subsection A.1, other factors may play a role in the selection of a candidate intervention for inclusion into the pool of available candidate interventions. For instance, it may be preferred or required that a candidate intervention satisfy a prescribed popularity condition. Different ways of assessing popularity are possible. In one technique, an administrator (or automated agent) can identify the number of times that a population of users has performed a particular type of activity, such as by performing a particular kind of task on a social networking system or the like. If the frequency measure exceeds a prescribed implementation-specific threshold, then the administrator may regard that type of activity as suitably popular. In addition, or alternatively, an administrator (or automated agent) can assess the popularity of a type of activity based on the frequency at which the specific user under consideration (who is the target of the intervention) engages in that type of activity. As also mentioned above, the administrator (or automated agent) may also favor activities that are relatively simple to perform, by requiring or preferring that each candidate intervention satisfy a simplicity condition.

The pool of interventions may also include a subset of interventions that do not meet one or more factors specified above. For example, some candidate interventions may correspond to types of techniques that are specifically developed to address psychological issues, and serve no general-purpose and familiar pre-existing uses. For example, one such special-purpose tool may guide the user in establishing a desired breathing pattern to reduce the user's level of stress.

A.3. Functionality for Choosing Among the Candidate Interventions

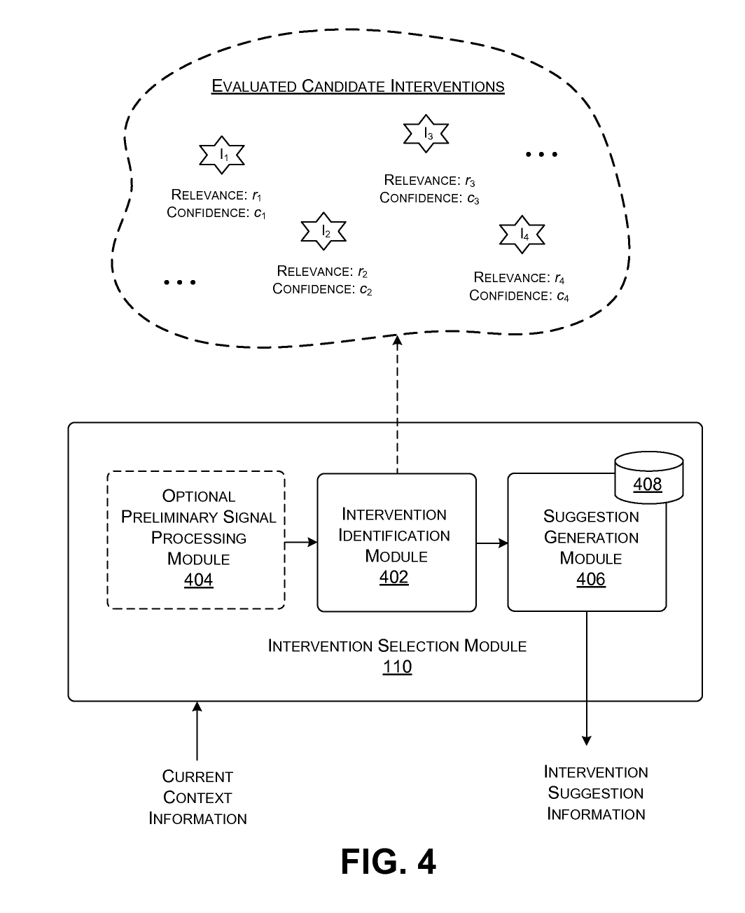

FIG. 4 shows one implementation of an intervention selection module 110, introduced in the context of FIG. 1. From a high-level perspective, in one non-limiting implementation, the intervention selection module 110 receives context information which describes the context in which an intervention is to be generated, with respect to a current time and a particular user. The intervention selection module 110 then maps the context information to one or more recommended interventions. The intervention selection module 110 then formulates intervention suggestion information which describes the one or more recommended interventions. The intervention selection module 110 can also optionally rank the recommended interventions; in that case, the intervention suggestion information also conveys ranking information, such as by ordering the recommendations by relevance, and/or by conveying relevance in some other manner.

In one implementation, the intervention selection module 110 can use a model-driven intervention identification module 402 to identify the recommended interventions, applying the model 106 produced by machine learning. The intervention identification module 402 can use any technology to perform this task, such as by using a regression tree, a classification tree, an ensemble of regression or classification trees (e.g., as formulated as a random forest or some other configuration), a neural network, a linear model, etc., or any combination thereof.

In another approach, the intervention selection module 110 can optionally include a preliminary signal processing module 404. As the name suggests, the preliminary signal processing module 404 performs preliminary analysis on the context information. For example, the preliminary signal processing module 404 can analyze any of a voice signal, electrodermal signal, video signal, etc., to determine whether these signals exhibit stress in the user. In one implementation, for instance, the preliminary signal processing module 404 may use a model produced by machine learning to classify the input signal(s); the classification, for instance, identifies whether these signals exhibit stress. The output of the preliminary signal processing module 404 constitutes processed context information, which, in turn, serves as another component of the input information fed to the intervention identification module 402. The preliminary signal processing module 404 is optional in the sense that the analysis performed by that module can be alternatively integrated into analysis performed by the intervention identification module 402 itself, thus eliminating the use of a separate preliminary analysis stage.

In one implementation, the intervention identification module 402 models the selection of recommended interventions as a contextual multi-arm bandit problem. In that framework, the intervention identification module 402 is faced with the prospect of choosing the most appropriate candidate interventions from the pool of identified candidate interventions. However, at the time of prediction, the intervention identification module 402 typically has incomplete knowledge regarding the statistical effectiveness of each candidate intervention in the pool. In some cases, for instance, the intervention identification module 402 may be able to predict the relevance of a candidate intervention with a high degree of confidence because that intervention has been applied in many prior circumstances that resemble the present circumstance, and the success of that intervention has been recorded in each such prior instance. In other cases, the intervention identification module 402 may have considerably less information to judge the effectiveness of a candidate intervention; this may be due, for example, to the fact that the candidate intervention has been newly added to the pool of available candidate interventions (and thus lacks historical evidence regarding its prior success), and/or the particular circumstance that is now encountered is relatively uncommon. The intervention identification module 402 can address the above situation by predominately exploiting successful and well-proven candidate interventions. However, by using this strategy, the intervention identification module 402 may neglect a low-confidence candidate intervention that may prove, if chosen, to be more effective than the high-confidence interventions.

To address the above situation, the intervention identification module 402 adopts a balance between an exploitation mode and an exploration mode when choosing interventions. In the exploitation mode, the intervention identification module 402 places primary emphasis on the selection of candidate interventions having relatively high confidence values associated therewith, and which have proven successful in achieving desired therapeutic results. In the exploration mode, the intervention identification module 402 also places emphasis on the selection of candidate interventions having lower confidence values, thus “trying out” these untested interventions. In one implementation, whether a confidence value is considered “low” or “high” can be assessed by comparing the confidence level to one or more implementation-specific thresholds. An exploitation/exploration setting or configuration may determine the extent to which the intervention identification module 402 chooses the exploitation mode over the exploration mode.

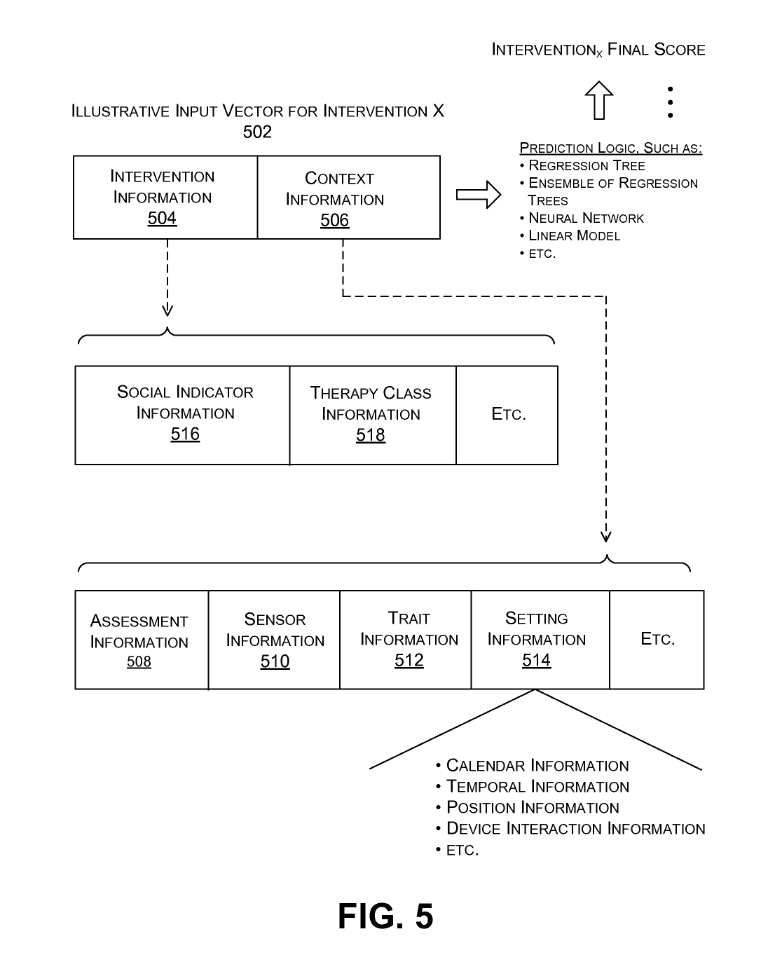

The intervention identification module 402 can use different techniques to balance the exploitation mode with the exploration mode. Consider the non-limiting and illustrative case in which the model generating module 104 (of FIG. 1) produces at least one regression tree based on the training data in the data store 108. The regression tree includes a hierarchy of nodes which terminate in a set of leaf nodes. To apply such a tree, the intervention identification module 402 generates an input vector, made up of feature values which describe a candidate intervention and the context information (to be described further below with reference to FIG. 5). The feature values in the input vector define a particular path through the tree, which terminates in a particular leaf node. That leaf node identifies a relevance score (r) associated with the candidate intervention. That leaf node can also identify the confidence level (c) associated with the candidate intervention. Or the intervention identification module 402 may generate the confidence level based on other information, such as by analyzing the variance in relevance scores provided by an ensemble of regression trees, for the intervention under consideration.

In one implementation, the intervention identification module 402 can select a final score for the intervention under consideration by modifying the original relevance score (r) by an upper bound defined by the confidence level (c) associated with this intervention. For example, if the original relevance score is 0.5 and the confidence level is ±0.1, then the intervention identification module 402 can choose a final score of 0.6 for this candidate intervention. This strategy leverages the exploitation model insofar as it bases the final score on the original relevance score (r), which, in turn is based on historical evidence of prior success. At the same time, the strategy also leverages the exploration mode by elevating the relevance score as a positive function of the uncertainty level, thereby “exploring” interventions lacking sufficient historical evidence of prior success. Alternatively, the intervention identification module 402 can apply a weighting factor to control the degree to which the confidence level (c) influences the offsetting of the original relevance score (r).

The intervention identification module 402 can apply yet other techniques to select a balance between the exploitation mode and the exploration mode. In another case, for instance, the intervention identification module 402 can use the original relevance score (r), by itself, to choose the recommended interventions for x % of the selections that are made, thus leveraging the exploitation mode over the exploration mode. In the remainder of the selections (100-x %), the intervention identification module 402 can randomly select an intervention from the pool of candidate interventions, thus leveraging the exploration mode over the exploitation mode. The value of x can be selected to satisfy any implementation-specific performance objective. For example, consider the case in which x is 80. For this setting, the intervention identification module 402 will select candidate interventions 80% of the time based primarily on the relevance score criterion, thus potentially ignoring uncertain candidate interventions with lower relevance scores (but which, if selected, might prove to be actually highly relevant). The intervention identification module 402 will randomly select interventions for 20% of the time without regard to their relevance scores; this makes it more likely that the intervention identification module 402 will select uncertain interventions with lower relevance scores, and thereby explore the space of uncertain interventions. The extent of exploration may be increased by decreasing x to achieve any implementation-specific performance objective.

Whatever technique is used to handle the above-described balance, the intervention identification module 402 may determine a final score for each intervention. The intervention identification module 402 may perform this task by generating and processing an input vector associated with each intervention, in successive or parallel fashion. The intervention identification module 402 then picks the single intervention having the highest score, or the set of candidate interventions having the highest scores.

The intervention selection module 110 can take into consideration other factors in choosing recommendations. For instance, in one implementation, the intervention selection module 110 also attempts to introduce novelty into the selection of recommended interventions. The intervention selection module 110 can achieve this goal in different ways. In one approach, the intervention identification module 402 can prepare an input vector having at least one feature value that describes the frequency at which a candidate intervention has been selected in a recent prior window of time. The intervention identification module 402 can then use this frequency value as a discounting factor, causing the intervention identification module 402 to disfavor the intervention as a direct function of its frequency of prior use. In another implementation, the intervention identification module 402 can select the n top-ranked candidate interventions without reference to their novelty, but then suitably discount each of the n candidate interventions by its respective frequency of prior use. Alternatively, or in addition, the intervention identification module 402 can regenerate the model used by the intervention identification module 402 on a relatively frequent basis, based on newly acquired context information; presuming that the context information changes over time, this updating tactic may cause, in some instances, the intervention identification module 402 to select fresh candidate interventions after the model is updated. Alternatively, or in addition, an administrator or automated agent can supply additional candidate interventions to the pool of candidate interventions; this tactic may increase the variety of interventions from which to choose.

The intervention selection module 110 can also use different strategies to identify interventions that are appropriate to particular respective users. In one approach, the intervention selection module 110 can achieve this goal by using a single model that effectively describes many different types of people having different respective characteristics. For example, the model generating module 104 can produce a regression tree model having different branches associated with different types of people. In another case, the intervention selection module 110 can train and apply different respective models for different individual users, or different classes of users. In another implementation, the intervention selection module 110 can produce a generic model that applies to all users, and then train a collection of models that are appropriate to different respective users or classes of users. A final score in this last-mentioned case may be produced by combining a score provided by the generic model with a score produced by an appropriate individual model. The intervention selection module 110 can employ yet other techniques to take differences among users into account.

In another case, the model generating module 104 can produce one or more models that target that segment of the user population which is most needful and desirous of receiving interventions. This strategy is based on the assumption the interventions will be most useful and/or effective for this segment of the population. Further, the predictive accuracy of these models can be improved by eliminating training data associated with groups outside the above-described target user population. Users who fall within the target population may be discriminated from other users by context information, such as user trait information, sensor information, etc.

As a point of clarification, the intervention selection module 110 was described above in the particular context of a contextual multi-arm bandit framework. But the principles set forth herein can be extended to other approaches, such as other reinforcement learning technology, collaborative filtering technology, learning-to-rank technology, etc. Other implementations can make recommendations using other tools, such as artificial intelligence rules-based techniques, etc.

Finally, the intervention suggestion module 110 also may include a suggestion generation module 406. The suggestion generation module 406 formulates intervention suggestion information which expresses the chosen interventions as one or more messages. For example, the messages can adopt the non-limiting format shown in the last column of FIG. 3. The suggestion generation module 406 then forwards the intervention suggestion information to the one or more user devices 210 via the computer network 216. The suggestion generation module 406 can consult a data store 408 of stock message content when constructing the intervention suggestion information. The intervention suggestion module 110 may also optionally convey the degree of relevance of each recommended intervention, such as by ranking the recommended interventions based on their final relevance scores. The intervention suggestion module 110 may also optionally convey information that identifies a time at which the recommended intervention(s) are to be revealed to the user and/or automatically invoked. In other words, the delivery of an intervention does not necessarily coincide with the time that it is revealed to the user or invoked; revelation and invocation may be delayed for any reason.

FIG. 5 shows an illustrative input vector 502 that the intervention identification module 402 may prepare and subsequently analyze. A first part of the input vector 502 corresponds to intervention information 504, which describes an intervention under consideration. A second part of the input vector 502 corresponds to context information 506, which describes the context in which the intervention under consideration is to be applied to a particular user at a current time in a particular setting.

Referring first to the context information 506, a first item in this information corresponds to current mood assessment information 508 (“assessment information” for brevity). The assessment information 508 may describe a user's optional self-assessment of his or her mental state. The assessment information 508 can be expressed in any manner, such as a value within a prescribed range of values, or a location or vector within a multi-dimensional representation of mental state. In the manner described in the next subsection, the user interaction mechanism 114 may allow the user to manually input this self-assessment information by interacting with a graphical user interface presentation, or through any other interface mechanism.

A second item of the context information 506 corresponds to sensor information 510, provided by one or more body sensing mechanisms. More specifically, the sensor information 510 includes information provided by physiological sensing mechanisms, voice analysis mechanisms, eye gaze detection mechanisms, gesture recognition mechanisms, and so forth.

A third item in the context information 506 corresponds to user trait information 512. The user trait information 512 represents the personality-related characteristics of the user, including mental health issues from which the user may suffer. A user may provide this information prior to first using the computer system 102, and/or periodically thereafter (e.g., on a monthly or yearly basis thereafter). In one technique, the user may provide the user trait information 512 by filling out one or more personality-related questionnaires. Alternatively, or in addition, the intervention selection module 110 can automatically infer the user trait information 512 based on information that it extracts, while complying with applicable privacy considerations and expectations, from available sources; such information can include demographic information regarding the user (age, gender, education level, place of residence, etc.), online habits exhibited by the user, online purchases made the user, and so forth. The intervention selection module 110 may harvest this information at any frequency.

A third item of the context information 506 corresponds to setting information 514. The setting information 514 describes the contextual setting in which the identified candidate intervention is to be delivered. The setting information 514, in turn, includes various items of component information.

Temporal-related information. For example, the setting information 514 may include temporal-related information, such as calendar information and time information. The calendar information may characterize the degree of busyness of a person, based on the number of upcoming entries in the person's calendar. Another measure may identify the amount of time until a next event is to occur in the person's schedule, such as a meeting. The time information may identify the date and time of day. The time information may also characterize the time of day, e.g., by indicating that it correspond to nighttime, mealtime, etc.

Position information. The setting information 514 may also include position information which describes the current position of the user, e.g., as provided by GPS technology and/or some other position-detection technology. For example, the position information may provide an indication of a number of readings that are received at various reference locations, such as the user's home or workplace. Assuming that the readings are received at regular intervals, the number of readings indicates the amount of time that the user has recently spent at these locations. The position information may also provide relative location information, such as by indicating the distance that the user is from different reference locations, such as user's home or workplace. The position information may also provide an indication of the amount of time that has transpired since the user has visited certain reference locations, such as the user's home or workplace. The position information may also provide an indication of a degree to which the use is moving about, as reflected by the diversity of position readings within a prescribed timeframe, and so on.

Device interaction information. The setting information 514 may also convey device interaction information, reflecting the manner in which the user has been interacting with his or her user devices 210 over some recent window of time. For example, the device interaction information may indicate the extent to which the user has moved a mobile user device, as measured by the accelerometers and/or gyroscopes provided by the mobile user device. The device interaction information can also characterize the nature of those movements, e.g., whether they are predominately slow and fluid, or quick and jerky. The device interaction information may also characterize the number of times that the user has performed certain actions on the device, such as the number of times that the user has unlocked the screen, or the number of times that the user has used certain applications, or the number of times that the user has performed certain computer-related actions within those applications, and so on.

Environmental information. The setting information 514 can also include contextual information that pertains to the environment in which the user operates, but may not directly relate to attributes or actions associated with the individual target user. For example, the setting information can describe aspects of the weather, financial markets, traffic patterns, airport delays, etc. The setting information 514 may also reflect statistical conclusions that have been derived by examining the traits and habits of groups of people, such as a conclusion that many people experience a high amount of stress when commuting to and from work.

Confidence information. The setting information 514 can also include confidence information which describes the level of confidence associated with any of the above-described measures. For example, the confidence information can provide an indication of the degree of reliability of the position data collected over a prescribed timeframe.

The intervention information 504 may likewise be composed of different items of component information, each of which describes a different aspect of the candidate intervention under consideration. A first item of information corresponds to social indicator information 516. The social indicator information 516 indicates whether the candidate intervention is typically performed by the user in solitary fashion, or by the user in conjunction with one or more other people. For example, an activity which entails accessing and viewing a cartoon is typically a solitary activity, while an activity which involves communicating with a friend is a social activity. More specifically, the social indicator information 516 may include a flag which is toggled on or off depending on the solitary/non-solitary nature of the intervention under consideration.

A second item of intervention information 504 corresponds to therapy class information 518. The therapy class information identifies the class (or classes) or therapy associated with the candidate intervention. In the simplified context of FIG. 3, the therapy class information 518 may identify whether the candidate intervention is associated with the positive psychology, cognitive behavioral, meta-cognitive, or somatic classes.

The intervention information 504 and context information 506 may include yet of items of information, although not shown in FIG. 5. For example, in another implementation, the intervention information 504 can include frequency information which identifies the number of times that the candidate intervention has been chosen within some recent window of time. Alternatively, or in addition, a particular implementation can omit one or more items of information described above.

As noted above, the intervention identification module 402 can feed the input vector 502 into one or more models. The model may map the input vector into a relevance score (r) that identifies the estimated effectiveness of the candidate intervention to the user, in his or her current circumstance. In the case of an ensemble of trees, the intervention identification module 402 can produce a relevance score by averaging the relevance scores provided by the individual trees in the ensemble. In addition, the model may optionally provide a confidence measure (c) that identifies a level of confidence associated with the relevance score.

In other implementations, the intervention selection module 110 can operate with a reduced reliance on the contextual information. For example, in one case, the intervention selection module 110 entirely ignores all contextual information, e.g., by presenting interventions in a random manner, without making reference to the particular situation that may apply to an individual target user. In another case, the intervention selection module 110 can generate interventions that take into account contextual information that affects all users, or large numbers of users, but without consideration of the specific circumstance that may affect the target user. For example, the intervention selection module 110 can make note of the time of day (adjusted by time zone), and then generate recommended interventions that most users find useful for that time of day. In another case, the intervention selection module 110 can observe that there is a sharp decline in the global stock market, or some other unfavorable news-related event. In response, the intervention selection module 110 may send recommended interventions to the user, under the assumption that such an event is likely to cause stress. In yet other cases, the intervention selection module 110 can produce recommendations by making reference to only some user-specific context information, but not other user-specific context information. For example, the intervention selection module 110 may omit the protocol by which it explicitly asks the user to rate his or her own mood; but the intervention selection module 110 may still collect context information provided by one or more sensing mechanisms. In other cases, the intervention selection module 110 can collect self-assessment information but not sensor information, and so on.

A.4. Functionality for Delivering the Intervention Suggestion Information

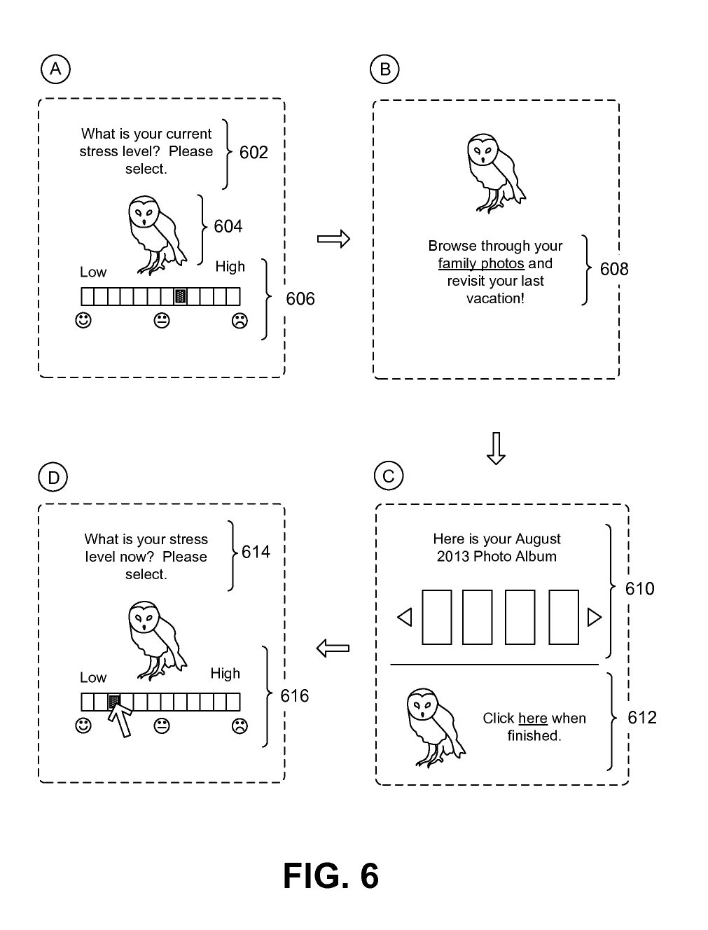

FIG. 6 shows one illustrative flow that the computer system 102 (of FIG. 1) may use to interact with the user, e.g., by soliciting input from the user and providing intervention suggestion information to the user. In one implementation, the user interaction mechanism 114, in cooperation with the intervention selection module 110, provides the user experience shown in FIG. 6.

In one implementation, the computer system 102 provides the illustrative flow as a sequence of graphical interface presentations. The computer system 102 may present these graphical interface presentations on any user device, such as the user's smartphone. In addition, or alternatively, the computer system 102 can formulate and present any aspect of the flow using other types of media content, such as audio messages, haptic information, and so on.

In state (A), the computer system 102 presents a message 602 which optionally invites the user to assess their current stress level, or other psychological state. The computer system 102 may provide the message 602 in response to various triggering circumstances described above. To repeat, in one case, the computer system 102 may provide the message 602 when the user expressly requests an intervention. In another case, the computer system 102 provides the message 602 on a periodic basis or based on any specified fixed schedule, or on a random basis, or whenever the user performs some other action, such as by opening an application, unlocking a screen, etc. In another case, the computer system 102 provides the message 602 when it senses, based on the automatically collected context information, that the user is in need of an intervention (which, in turn, may be based on user-specific and/or user-agnostic considerations), and so on.

In some implementations, the intervention selection module 110 may also take into consideration override information that has the effect of overriding the generation or transmission of interventions. Or the override information may govern the mode of delivery that is used to transmit the candidate intervention information. For example, the override information may cause the intervention selection module 110 to refrain from sending recommended interventions during the nighttime (taking into account time zone), based on the assumption that the user is likely sleeping. Alternatively or in addition, the intervention selection module 110 may make reference to user-specified blackout periods (which may be stored in a user profile), for which it will not send recommended interventions. In other cases, the override information may cause the intervention selection module 110 to refrain from sending recommended interventions when it determines that the user is driving a vehicle. Or it may send the recommended interventions in audio form in this circumstance, not visual, so as to not distract the user while driving.

The computer system 102 may also optionally present an avatar 604 of any type, such as, in the non-limiting case of FIG. 6, an owl character. The computer system 102 may represent the avatar 604 to help the user quickly identify the nature of the flow that is being invoked by the computer system 102, and to promote a user-friendly user experience.

In some implementations, the computer system 102 collects self-assessment information. In those cases, the computer system 102 may also present a graphical control element 606 by which the user may rate his or her stress or other psychological state. In the case of FIG. 6, the graphical control element 606 corresponds to a graduated bar on which the user may rank his or stress, on a linear scale from low to high. In other cases, the graphical control element can be expressed in other formats, such as a radio buttons, menu selection, a multi-axis position selection, and so on. Alternatively, or in addition, the user may provide his or her input via other modes of interaction, such as via voice interaction, free-space gestures, etc. The computer system 102 may also use automatically collected sensor information to determine the user's current psychological state.

In some implementations, after the user assesses his or her mood, the intervention selection module 110 generates intervention suggestion information in the manner described above, e.g., by optionally collecting all of the context information described above and then mapping the context information into one or more recommended interventions. The intervention selection module 110 then delivers one or more messages to the user's user device, which convey the intervention selection information.

State (B) reflects the outcome of the above-described operation. Here, the intervention selection module 110 has generated a message 608 which invites the user to visit a website that allows the user to store personal photographs and other documents. The message specifically encourages the user to “Browse through your family photos and revisit your last vacation!” The theory behind this intervention is that the user's photographs will have a calming effect on the user, e.g., by transporting the user from his or her current stressful situation to a more pleasant, time and place. The message 608 may include a hyperlink which constitutes an activation mechanism by which the user may access the website. Alternatively, the message 608 may provide a separate URL or other kind of link to the website. In other cases, the message 608 may convey two or more recommended interventions. The message 608 can also order the interventions based on their final relevance scores.

Presume that the user activates the activation mechanism. As indicated in state (C), a provider computer system associated with the website responds by displaying the user's photographs 610. The computer system 102 may also display a message 612 which invites the user to indicate when he or she has finished performing the intervention, which, in this case, corresponds to viewing his or her vacation photos. The user may be expected to be familiar with the general type of activity associated with this intervention (although perhaps not the specific task of searching his or her photos for vacation-related content). Further, this type of activity has generally been performed for non-therapeutic reasons in the past.

In state (D), the computer system 102 may display a message 614 which invites the user to again rate his or her level of stress or other psychological state. The computer system 102 also presents a graphical control element 616, through which the user may input the self-assessment information. Alternatively, or in addition, the computer system 102 may use automatically collected sensor information to determine the user's current psychological state. The model generating module 104 may then use the pre-intervention and post-intervention stress information (collected in states A and D, and/or elsewhere) to retrain the model 106, at an appropriate juncture.

Although not illustrated in FIG. 6, the computer system 102 can also administer any reward system that encourages the users to interact with the computer system 102. For example, the reward system 102 may assign a number of points to the user when the user enters self-assessment information, and/or performs an intervention, and/or performs any other activity associated with the delivery of interventions. In one implementation, the user may redeem the reward points to receive various real-world benefits, such as good or services.

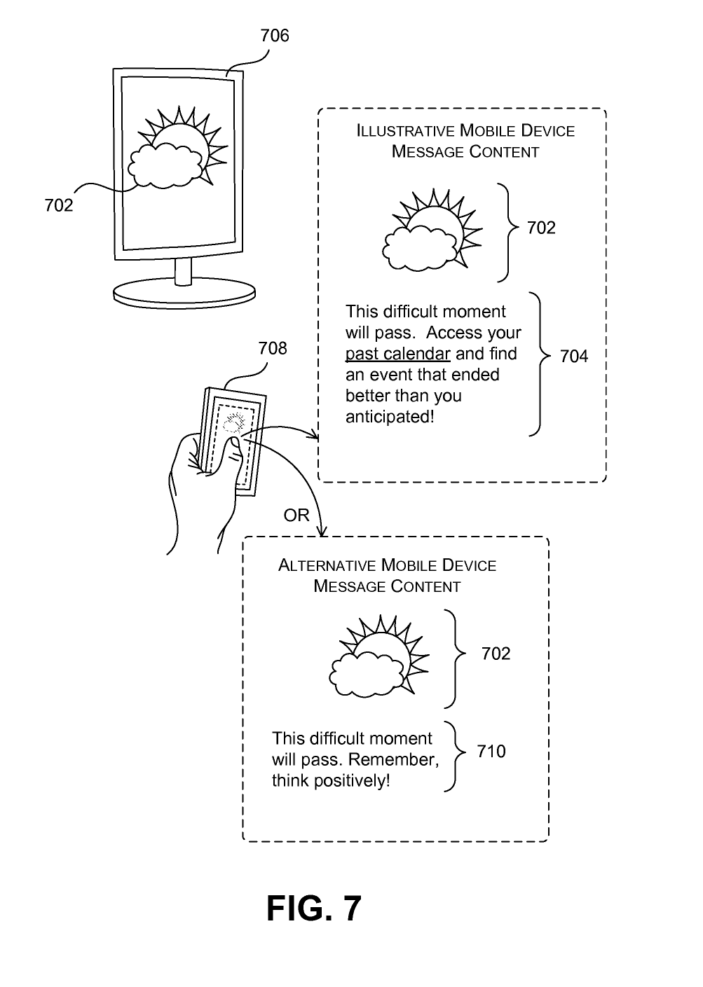

FIG. 7 shows an alternative manner by which the computer system 102 may express and deliver intervention suggestion information. In this case, the intervention selection module 110 formulates the intervention suggestion information as two messages. A first message provides ambient presentation information that represents the recommended intervention. A second message provides the ambient presentation information, in combination with explanatory content which describes the recommended intervention. Alternatively, or in addition, the first message can represent the ambient presentation information in non-visual format, such as via audio information, haptic information, etc. Likewise, the second message can, alternatively, or in addition, represent the second message in non-visual form, such as by presenting both the ambient presentation information and explanatory description in audio format.

In any event, the intent of the implementation of FIG. 7 is to omit or reduce the symbolic explanatory information in the first message, relative to the second message, which has the effect of concealing or obscuring the meaning of the first message. This implementation is useful in those cases in which the user wishes to hide or obscure the meaning of the intervention from observers—where an observer corresponds to any person who is not a target of the intervention. For example, if the observer is a child, the user may wish to prevent the child from learning the meaning of the intervention to avoid hurting the feeling of the child. In other cases, the user may wish to prevent an observer from learning the meaning of the intervention to protect the user's privacy.

In the example of FIG. 7, the first message provides a picture 702 of a sun emerging from behind a cloud. The second message provides the picture 702 in combination with explanatory content 704 which explains the intervention. In a first implementation, assume that the intervention encourages the recipient to look at a calendar application to find an event that ended with a positive outcome, even though the user may have feared the worst preceding the event. The intervention is intended to refute pessimism in the user, which can be classified as an intervention employing a positive psychology-type strategy, or a cognitive behavioral-type strategy, etc.

The intervention selection module 110 may present the first message on a first user device 706, and present the second message on a second user device 708. For example, the first user device 706 may correspond to the display monitor associated with a stationary personal computing device, a tablet-type device, and so on. The second user device 708 may correspond to a mobile device, such as a smartphone. In many implementations, the assumption is that the first user device 706 will have a larger display surface than the second user device 708, although this need not be so. Further, there may be an expectation that the display content of the first user device 706 is less private than the display content of the second user device 708 (e.g., depending on the sizes and placements of these two devices), although, again, this need not be so.

The bottom panel in FIG. 7 indicates that the second message can include a simplified intervention message that does not include an activation mechanism. Rather, the explanatory content 710 in this instance encourages the user as follows: “This difficult moment will pass. Remember, think positively!” In other words, that message does not leverage any computer-related resources in the manner described above.

In terms of user experience, when the first and second messages are sent, the user may first notice the picture 702 that appears on the first user device 706. The user may then consult the second message that appears on the second user device 708, which explains the intervention associated with the picture 702. After repeated encounters with these pair of messages, the user will likely remember the association between the picture 702 and a particular intervention. At that time, the user may no longer need to consult the second user device 708 to read the textual description provided by the second message.

The above-described mode receiving intervention information may appeal to the user for various reasons. First, as mentioned above, this mode protects the privacy of the user, and reduces the chances of offending any observer who is not the target of the intervention. Second, the user may find it more convenient to view the picture on the first user device 706, rather than pick up and interact with the second user device 708, especially when the user's is otherwise occupied with other tasks, such as cooking, caring for children, interacting with co-workers, etc.

In another implementation, the intervention selection module 110 can modulate one or more visual aspects of the picture 702 to convey additional information. For example, the intervention selection module 110 can modulate the size, color, motion dynamics, etc. of the picture 702 to convey an urgency level associated with the intervention or any other aspect of the intervention. If the ambient presentation corresponds to audio information, the intervention selection module 110 can modulate the volume and/or other aspects of this audio presentation.

B. Illustrative Processes

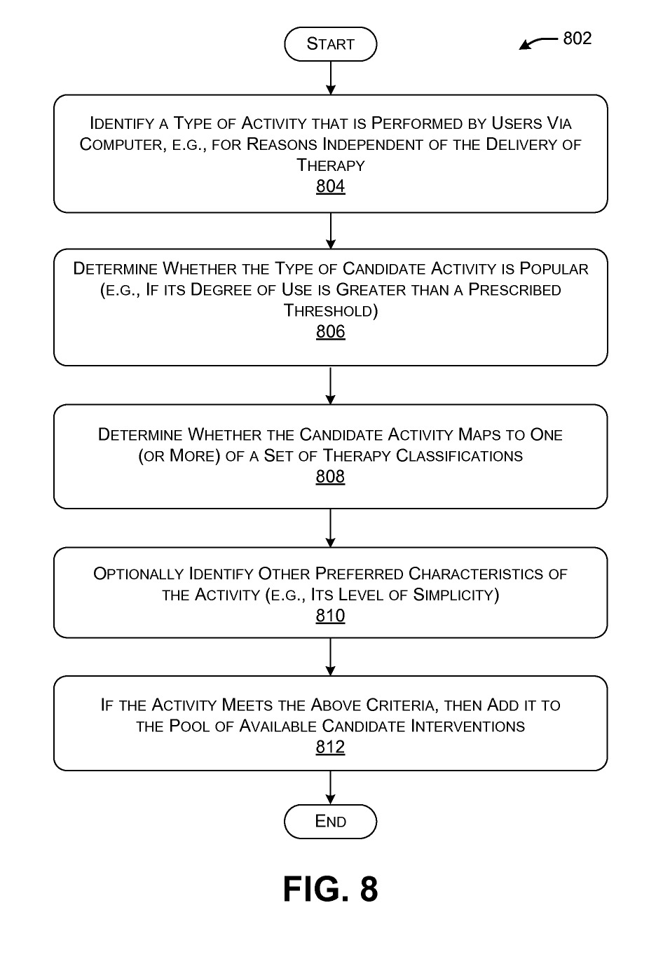

FIGS. 8-13 show procedures that explains one manner of operation of the computer system 102 of Section A. Since the principles underlying the operation of the computer system 102 have already been described in Section A, certain operations will be addressed in summary fashion in this section.

To begin with, FIG. 8 shows a procedure 802 for selecting some or all of the candidate interventions for inclusion in the pool of candidate interventions. One or more human administrators can perform this procedure 802. Alternatively, or in addition, an automated agent can perform at least some aspects of the procedure 802.

In block 804, the person or agent identifies a type of activity that is performed by users using one or more computing devices, in an online mode, offline mode, or a combination of online and offline modes. The users have performed this type of activity for a pre-existing purpose that may be independent of the delivery of therapy. For example, an activity type that relates to the use of an Email system may have been performed for the primary purpose of communication per se, not therapy.

In block 806, the person or agent determines whether the type of candidate activity is considered popular. The person or agent can make this determination by determining whether the type of activity meets a prescribed popularity condition, such as whether its frequency of use is greater than a prescribed implementation-specific threshold.

In block 808, the person or agent determines whether the candidate activity maps to one or more of a set of therapy classifications. FIG. 3 provides a simplified list of four representative therapy classifications. For example, sending an Email message to a friend, asking the friend for advice, maps to the cognitive behavioral category set forth in FIG. 3.

In block 810, the person or agent determines whether the candidate activity meets other prescribed requirements or preferences. For example, the person or agent can determine whether the activity is suitably simple, as measured based on any metric of simplicity.

In block 812, the person or agent can add the candidate activity to the pool of available candidate interventions if it meets all of the criteria set forth above. In other cases, the person or agent can add the activity to the pool of available interventions even though it does not meet all the criteria; in this case, the person or agent may choose to negatively weight the activity to indicate that it is not fully satisfactory in one or more respects.

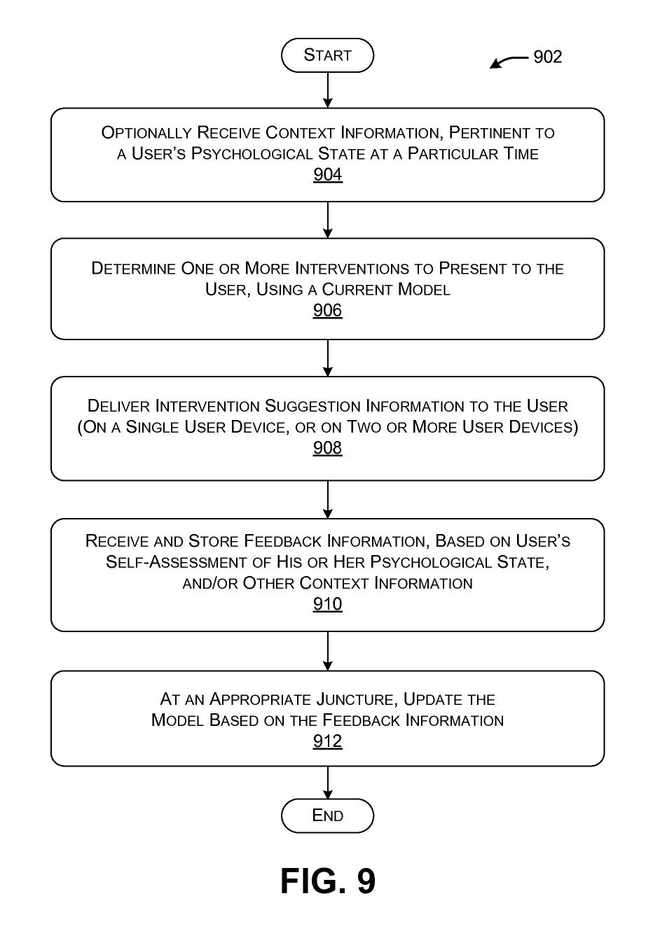

FIG. 9 shows a procedure 902 which describes an overview of one manner of operation of the computer system 102 of FIG. 1. In block 904, the computer system 102 optionally receives context information which has a bearing on the psychological state of the user at a particular time. FIG. 5 and the accompanying explanation (in Section A) set forth different items of information that may be expressed by the context information. The context information can encompass information that is specific to a particular target user and/or information that is relevant to all users or some class of users. In other implementations, the computer system 102 omits block 904.

In block 906, the computer system 102 determines one or more interventions to present to the user, based on the context information and/or other factors, through the use of a model 106. A model generating module 104 produces the model 106 in an offline fashion.

In block 908, the computer system 102 formulates and delivers intervention suggestion information to one or more user devices. The intervention suggestion information expresses the recommended interventions identified in block 906.

In block 910, the computer system 102 receives feedback information. The feedback information may optionally reflect the user's self-assessment of his or her psychological state before and after conducting the intervention. Alternatively, or in addition, the feedback information may include sensor information (and other automatically collected context information), collected at various junctures.

In block 912, the computer system 102 updates the model 106 based on the received feedback information. The computer system 102 may perform this task on any basis, such as a periodic basis, an event-driven basis, and so on.

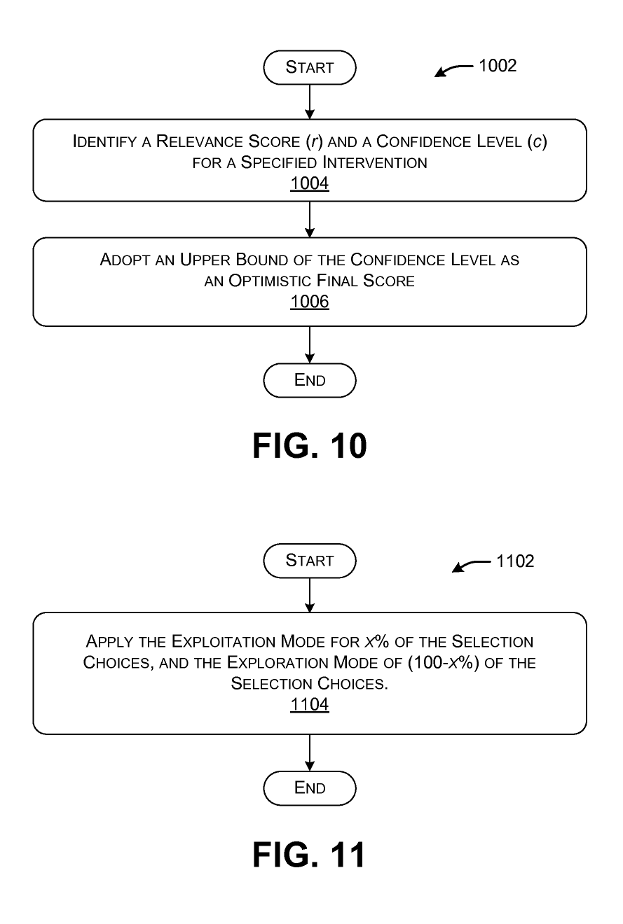

FIGS. 10 and 11 show two respective procedures (1002, 1102) by which the intervention selection module 110 (of FIG. 4) may balance an exploitation mode with an exploration mode in selecting interventions to present to a user. In block 1004 of FIG. 10, for instance, the intervention selection module 110 may identify a relevance score (r) and a confidence level (c) associated with a particular candidate intervention under consideration. In block 1004, the intervention selection module 110 can select an upper bound of the confidence level as a final score. For example, if the relevance score value is x and the confidence level is ±y, then the final score corresponds to x+y, or x+w*y, where w is some weighting factor. This strategy presents a particular balance between the exploitation mode and the exploration mode for reasons set forth in Section A.

In the alternative approach described in block 1104 of FIG. 11, the intervention selection module 110 applies an exploitation mode for every x % of the candidate intervention choices it makes. The interventions selection module 110 applies the exploration mode for (100-x %) of choices it makes. In the exploitation mode, the intervention selection module 110 may choose one or more candidate interventions based on only their relevance scores. In the exploration mode, the intervention selection module 110 may choose the candidate interventions in a random fashion.

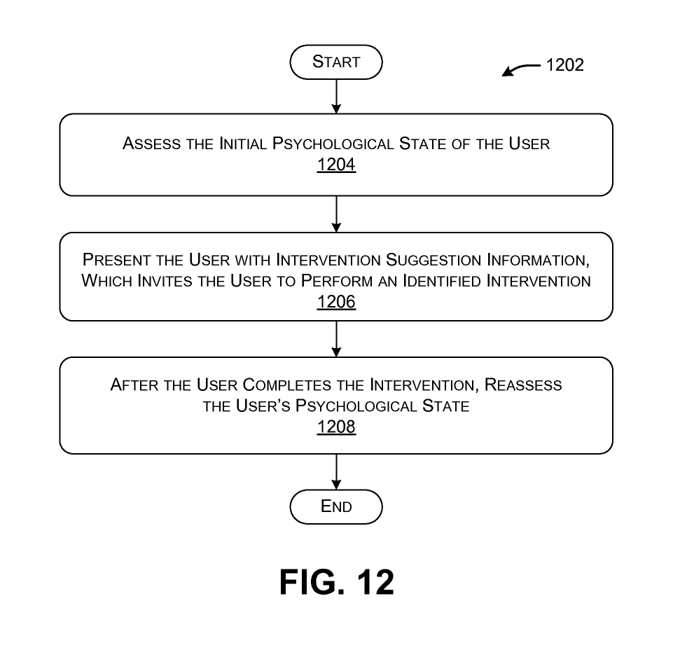

FIG. 12 shows a procedure which describes one interaction flow that may be provided by the computer system 102 of FIG. 1, corresponding to the example of FIG. 6. In block 1204, the computer system 102 invites the user to assess their mood. The computer system 102 then collects the self-assessment information entered by the user in response thereto. In block 1206, the computer system 102 presents intervention suggestion information to the user, which invites the user to perform an identified intervention. In block 1204, the computer system 102 again collects self-assessment information after the user performs the intervention. The computer system 102 may alternatively, or in addition, automatically collect pre-intervention and post-intervention mood information based on sensor information provided by one or more sensing mechanisms, etc.

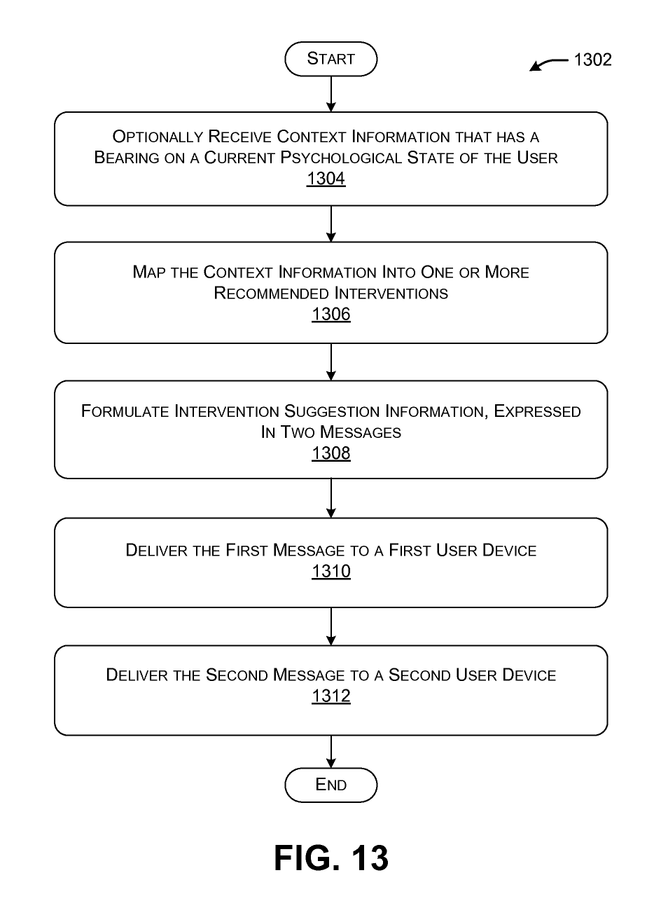

FIG. 13 shows a procedure 1302 which represents an alternative mode of delivering intervention suggestion information to two user devices, corresponding to the example of FIG. 7. In block 1304, the computer system 102 optionally receives context information that has a bearing on the current psychological state of the user. In block 1306, the computer system 102 maps the context information into one or more recommended interventions, or generates the recommended interventions based on some other consideration or combination of considerations.

In block 1308, the computer system 102 formulates intervention suggestion information, which identifies the one or more recommended interventions. More specifically, the intervention suggestion information includes two messages. A first message provides an ambient presentation relating to a recommended intervention. A second message provides the ambient presentation in conjunction with a textual message that explains the recommended intervention.

In block 1310, the computer system 102 delivers the first message to a first user device. In block 1312, the computer system 102 delivers the second message to a second user device.

C. Representative Computing Functionality

FIG. 14 shows computing functionality 1402 that can be used to implement any aspect of the computer system 102 of FIG. 1. For instance, the type of computing functionality 1402 shown in FIG. 14 can be used to implement any aspect of the intervention computer system 204 of FIG. 2, any aspect of the provider computer systems (206, 208, . . . ) of FIG. 2, and/or any aspect of the user devices 210 of FIG. 2, etc. In all cases, the computing functionality 1402 represents one or more physical and tangible processing mechanisms.

The computing functionality 1402 can include one or more processing devices 1404, such as one or more central processing units (CPUs), and/or one or more graphical processing units (GPUs), and so on.

The computing functionality 1402 can also include any storage resources 1406 for storing any kind of information, such as code, settings, data, etc. Without limitation, for instance, the storage resources 1406 may include any of: RAM of any type(s), ROM of any type(s), flash devices, hard disks, optical disks, and so on. More generally, any storage resource can use any technology for storing information. Further, any storage resource may provide volatile or non-volatile retention of information. Further, any storage resource may represent a fixed or removal component of the computing functionality 1402. The computing functionality 1402 may perform any of the functions described above when the processing devices 1404 carry out instructions stored in any storage resource or combination of storage resources.

As to terminology, any of the storage resources 1406, or any combination of the storage resources 1306, may be regarded as a computer readable medium. In many cases, a computer readable medium represents some form of physical and tangible entity. The term computer readable medium also encompasses propagated signals, e.g., transmitted or received via physical conduit and/or air or other wireless medium, etc. However, the specific terms “computer readable storage medium” and “computer readable medium device” expressly exclude propagated signals per se, while including all other forms of computer readable media.

The computing functionality 1402 also includes one or more drive mechanisms 1408 for interacting with any storage resource, such as a hard disk drive mechanism, an optical disk drive mechanism, and so on.

The computing functionality 1402 also includes an input/output module 1410 for receiving various inputs (via input devices 1412), and for providing various outputs (via output devices 1414). Illustrative input devices include a keyboard device, a mouse input device, a touchscreen input device, a digitizing pad, one or more video cameras, one or more depth cameras, a free space gesture recognition mechanism, one or more microphones, a voice recognition mechanism, any movement detection mechanisms (e.g., accelerometers, gyroscopes, etc.), any body sensing mechanisms, and so on. One particular output mechanism may include a presentation device 1416 and an associated graphical user interface (GUI) 1418. Other output devices include a printer, a model-generating mechanism, a tactile output mechanism, an archiving mechanism (for storing output information), and so on. The computing functionality 1402 can also include one or more network interfaces 1420 for exchanging data with other devices via a computer network 1422. One or more communication buses 1424 communicatively couple the above-described components together.

The communication network 1422 can be implemented in any manner, e.g., by a local area network, a wide area network (e.g., the Internet), point-to-point connections, etc., or any combination thereof. The communication network 1422 can include any combination of hardwired links, wireless links, routers, gateway functionality, name servers, etc., governed by any protocol or combination of protocols.

Alternatively, or in addition, any of the functions described in the preceding sections can be performed, at least in part, by one or more hardware logic components. For example, without limitation, the computing functionality 1402 can be implemented using one or more of: Field-programmable Gate Arrays (FPGAs); Application-specific Integrated Circuits (ASICs); Application-specific Standard Products (ASSPs); System-on-a-chip systems (SOCs); Complex Programmable Logic Devices (CPLDs), etc.

In closing, to repeat, the functionality described above can employ various mechanisms to ensure the privacy of user data maintained by the functionality, in accordance with user expectations and applicable laws of relevant jurisdictions. For example, the functionality can allow a user to expressly opt in to (and then expressly opt out of) the provisions of the functionality. The functionality can also provide suitable security mechanisms to ensure the privacy of the user data (such as data-sanitizing mechanisms, encryption mechanisms, password-protection mechanisms, etc.).

Further, the description may have described various concepts in the context of illustrative challenges or problems. This manner of explanation does not constitute a representation that others have appreciated and/or articulated the challenges or problems in the manner specified herein. Further, the claimed subject matter is not limited to implementations that solve any or all of the noted challenges/problems.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A computer system for providing intervention suggestion information, comprising:
an intervention selection module configured to:
generate intervention suggestion information, the intervention suggestion information identifying at least one recommended intervention that the user is invited to perform, with an objective of modifying a current psychological state of the user; and
deliver the intervention suggestion information to the user via one or more user devices,
said at least one recommended intervention being selected from a pool of available candidate interventions, each candidate intervention being chosen for inclusion in the pool such that the candidate intervention:
corresponds to a type of activity that has been performed using one or more computing devices;
corresponds to a type of activity that satisfies a prescribed popularity condition; and
maps to at least one therapy classification in a set of identified therapy classifications.

2. The computer system of claim 1, wherein said one or more user devices includes at least one mobile user device.

3. The computer system of claim 1, wherein at least one candidate intervention is performed using a social network system.

4. The computer system of claim 1, wherein at least one candidate intervention is performed using a message-sending system.

5. The computer system of claim 1, wherein at least one candidate intervention is performed using an online data storage system.

6. The computer system of claim 1, wherein the intervention selection module is further configured to:
receive context information, the context information describing a current context that applies to a user, and having a bearing on the current psychological state of the user; and
map the context information to the intervention suggestion information.

7. The computer system of claim 6, wherein the context information includes device interaction information which reflects a manner in which the user has been interacting with said one or more user devices.

8. The computer system of claim 6, wherein the setting information includes position information that describes a position of the user.

9. The computer system of claim 8, wherein the position information specifies the position of the user relative to one or more reference locations.

10. The computer system of claim 6, wherein the context information includes assessment information which describes a self-assessment, by the user, of a psychological state of the user prior to, and after, the user performs said at least one recommended intervention.

11. The computer system of claim 1, wherein the intervention selection module is configured to generate the intervention suggestion information without reference to user-specific context information.

12. The computer system of claim 11, wherein the intervention selection module is configured to generate the intervention suggestion information in a random manner.

13. The computer system of claim 1, wherein each candidate intervention in the pool of candidate interventions further satisfies a simplicity condition.

14. The computer system of claim 1, wherein the candidate suggestion information is formulated into two messages, including:
a first message, delivered to a first user device, which provides an ambient presentation relating to a recommended intervention; and
a second message, delivered to a second user device, which provides the ambient presentation in conjunction with explanatory content which describes the recommended intervention.

15. The computer system of claim 1, wherein the intervention selection module is configured to choose the intervention suggestion information based on a selected balance between an exploitation mode and an exploration mode,
wherein, in the exploitation mode, the intervention selection module is configured to select candidate interventions based primarily on respective proven levels of relevance of the candidate interventions,
and, in the exploration mode, the intervention selection module is configured to select candidate interventions by favorably weighting candidate interventions as a positive function of their respective levels of uncertainty.

16. A computer readable storage medium for storing computer readable instructions, the computer readable instructions providing an intervention selection module when executed by one or more processing devices, the computer readable instructions comprising:
logic configured to receive context information, the context information describing a current context that applies to a user, and having a bearing on a current psychological state of the user; and
logic configured to map the context information to intervention suggestion information, the intervention suggestion information identifying at least one recommended intervention that the user is invited to perform, with an objective of changing the current psychological state of the user,
wherein the logic configured to map is further configured to choose the intervention suggestion information based on a selected balance between an exploitation mode and an exploration mode,
wherein, in the exploitation mode, the logic configured to map selects candidate interventions based primarily on respective proven levels of relevance of the candidate interventions,
and, in the exploration mode, the logic configured to map selects candidate interventions by favorably weighting candidate interventions as a positive function of their respective levels of uncertainty.

17. The computer readable storage medium of claim 16, wherein the logic configured to map is configured to model selection of said at least one recommended intervention as a contextual bandit machine-learning problem.

18. The computer readable storage medium of claim 16, wherein said at least one recommended intervention is selected from a pool of available candidate interventions, each candidate intervention being chosen for inclusion in the pool such that the candidate intervention:
corresponds to a type of activity that has been performed using one or more computing devices, for a purpose that is independent of providing therapy;
corresponds to a type of activity that satisfies a prescribed popularity condition; and
maps to at least one therapy classification in a set of identified therapy classifications.

19. A method, performed by at least one computing device, for providing intervention suggestion information, comprising:
generating one or more recommended interventions that the user is invited to perform to change a current psychological state of the user;
formulating intervention suggestion information which expresses said one or more recommended interventions,
the intervention suggestion information including:
a first message that provides an ambient presentation relating to said one or more recommended interventions; and
a second message which provides the ambient presentation in conjunction with a message which identifies said one or more recommended interventions;
delivering the first message to a first user device; and
delivering the second message to a second user device.

20. The method of claim 19, wherein said one or more interventions are selected from a pool of available candidate interventions, each candidate intervention being chosen for inclusion in the pool such that the candidate intervention:
corresponds to a type of activity that has been performed using one or more computing devices, for a purpose that is independent of providing therapy;
corresponds to a type of activity that satisfies a prescribed popularity condition; and
maps to at least one therapy classification in a set of identified therapy classifications.
